(19) United States
(12) Patent Application Publication
(10) Pub. No.: US 2018/0308218 A1
(43) Pub. Date: Oct. 25, 2018

(54) NON-PARALLAX PANORAMIC IMAGING FOR A FLUOROSCOPY SYSTEM

(71) Applicant: Whale Imaging, Inc., Waltham, MA (US)

(72) Inventors: Changguo Ji, Lexington, MA (US); Paul Glazer, Chestnut Hill, MA (US); Xingbai He, Belmont, MA (US); William Wong, Milton, MA (US)

(21) Appl. No.: 15/496,879

(22) Filed: Apr. 25, 2017

(51) Int. Cl. G06T 3/40; A61B 6/00; G01N 23/04

(52) U.S. Cl. CPC G06T 3/4038; A61B 6/52; G01N 23/043; G06T 2207/10121; A61B 6/44; G06T 2207/30004; A61B 6/4233

(57) ABSTRACT

A method and system for creating non-parallax panoramic images from a plurality of individual images, in real-time is provided. Specifically, the present invention provides a system and method configured to combine individual overlapping medical images into a single undistorted panoramic image in real-time. In particular, the present invention provides a system and method for combining individual x-ray images into a single clinical panoramic image for use with a G-arm device.

### FIELD OF THE INVENTION

The present invention relates to an imaging system and method for producing a panoramic image for use during a fluoroscopic procedure. In particular, the present invention relates to fluoroscopic imaging system configured to create non-parallax panoramic images in real-time from a plurality of individual images captured by the imaging system.

### BACKGROUND

Generally, the usage of conventional C-arm X-ray equipment is well known in the medical art of surgical and other interventional procedures. Traditionally, the utilization of C-arm X-ray equipment enables flexibility in operation procedures and in the positioning process, which is reflected by a number of degrees of freedom of movement provided by the C-arm X-ray equipment.

In a conventional implementation, a C-arm gantry is slideably mounted to a support structure to enable orbiting rotational movement of the C-arm about a center of curvature for the C-arm. Additionally, the C-arm equipment provides a lateral rotation motion rotating along the horizontal axis of the support structure. Moreover, the C-arm equipment also can include an up-down motion along the vertical axis, a cross-arm motion along the horizontal axis, and a wig-wag motion along the vertical axis.

A traditional C-arm provides real time X-ray images of a patient's spinal anatomy which is used to guide a surgeon during an operating procedure. For example, spinal deformity correction is a type of surgery that frequently uses the C-arm during an operation procedure. Such surgeries typically involve corrective maneuvers to improve the sagittal or coronal profile of the patient. However, an intra-operative estimation of the amount of correction is difficult. Mostly, anteroposterior (AP) and lateral fluoroscopic images are used, but are limited as the AP and lateral fluoroscopic images only depict a small portion of the spine in a single C-arm image. The small depiction of the spine in traditional C-arm images is due to the limited field of view of a C-arm machine. As a result, spine surgeons are in need of an effective tool to image an entire spine of a patient for use during surgery and for assessing the extent of correction in scoliotic deformity.

Similarly, the full bone structure of a patient cannot be captured in a single X-ray image with existing digital radiography systems. Stitching methods and systems for X-ray images are very important for scoliosis or lower limb malformation diagnosis and pre-surgical planning. Although radiographs that are obtained either by using a large field detector or by image stitching can be used to image an entire spine, the radiographs are usually not available for intra-operative interventions because there are not motorized positioning mechanisms implemented for conventional digital radiography systems along a horizontal positioning of a patient.

One alternative to conventional radiographs is to develop methods and systems to stitch multiple intra-operatively acquired small fluoroscopic images together to be able to display the entire spine at once. Panoramic images are useful preoperatively for diagnosis, and intra-operatively for long bone fragment alignment, for making anatomical measurements, and for documenting surgical outcomes. There are existing methods to create a single panoramic image of a long view using C-arm from several individual fluoroscopic X-ray images. (See, U.S. Patent Application No. 2011/0188726). In particular, U.S. Patent Application No. 2011/0188726 discloses a method for generating a panoramic image of a region of interest (ROI) which is larger than a field of a view of a radiation based imaging device, including, positioning markers along the ROI, acquiring a set of images along the ROI, in which the acquired images have at least partially overlapping portions, aligning at least two separate images by aligning a common marker found in both images and compensating for a difference between a distance from a radiation source to the marker element and the distance from the radiation source to a plane of interest. Additionally, the stitching methods of traditional systems typically utilize image down-sampling and image mask to decrease the size of image and reduce the amount of computation.

Although the C-arm X-ray equipment is smart and flexible in positioning process, it is often desirable to take X-rays of a patient from both the anteroposterior and lateral positions (two perpendicular angles), in such situations, the operators have to reposition the C-arm because C-arm configurations do not allow for such perpendicular bi-planar imaging. For taking the X-rays from different angles at the same time without repositioning the X-ray apparatus, such a configuration is often referred to as bi-planar imaging, also known as G-arm or G-shape arm (see, U.S. Pat. No. 8,992,082), that allows an object to be viewed in two planes simultaneously. The two plane imaging is enabled by the utilization of two X-ray beams emitted from the two X-ray tubes crossing at an iso-center.

A traditional mobile dual plane fluoroscopy device has advantages of each of C-shaped, G-shaped, and ring-shaped arm configurations. The device consists of a gantry that supports X-ray imaging machinery. The gantry is formed to allow two bi-planar X-rays to be taken simultaneously or without movement of the equipment and/or patient. The gantry is adjustable to change angles of the X-ray imaging machinery. In addition, the X-ray receptor portion of the X-ray imaging machinery may be positioned on retractable and extendable arms, allowing the apparatus to have a larger access opening when not in operation, but to still provide bi-planar X-ray ability when in operation. With respect to providing real-time panoramic images for use during a fluoroscopic procedure with a G-arm device, the G-arm has similar shortcomings as discussed with respect to the C-arm.

### SUMMARY

There is a need for improvements to producing a panoramic image of a patient subject, in real-time, during a medical procedure. The present invention is directed toward further solutions to address this need, in addition to having other desirable characteristics. Specifically, the present invention provides a system and method configured to combine individual overlapping medical images into a single undistorted panoramic image in real-time. In particular, the present invention provides a system and method for combining individual x-ray images into a single clinical panoramic image for use with a C-arm or G-arm device for use during a fluoroscopic procedure.

In accordance with example embodiments of the present invention, a panoramic fluoroscopic imaging system is provided. The system includes a radiation source configured to output electromagnetic radiation, a radiation detector coupled to a motorized gantry stage and disposed to read electromagnetic radiation output by the radiation source, and a dynamic collimator coupled to the radiation source that focuses the electromagnetic radiation output by the radiation source and directs the focused electromagnetic radiation at the radiation detector, such that the radiation detector changes position based on a position of the motorized gantry stage. The system also includes a processing and display device in communication with the fluoroscopic imaging device. The processing and display device is configured to receive raw image data from the radiation detector, the raw image data includes a plurality of images captured at the position of the motorized gantry stage relative to a subject patient located between the radiation source and the radiation detector. The processing and display device also includes transforming the raw image data for each of the plurality of images into displayable images, stitching together the displayable images, based on the position of the motorized gantry stage, into a non-parallax panoramic image, and displaying the non-parallax panoramic image on the display device in real time.

In accordance with aspects of the present invention, the radiation detector includes a thin film transistor (TFT) flat-panel detector with a scintillation material layer. When the TFT receives energy from visible photons that charge capacitors of pixel cells within the TFT panel, charges from each of the pixel cells are readout as a voltage data value to the processing and display device.

In accordance with aspects of the present invention, the radiation detector includes an image intensifier configured to readout a voltage data value to the processing and display device. The single non-parallax panoramic image provides data for use during a fluoroscopic procedure. The radiation source, the radiation detector, the dynamic collimator, and the motorized gantry stage can all be disposed within a C-arm fluoroscopic system.

In accordance with aspects of the present invention, the system further includes a second radiation source, a second radiation detector coupled to a second motorized gantry stage, and a second dynamic collimator. The radiation source, the second radiation source, the radiation detector, the second radiation detector, the dynamic collimator, the second dynamic collimator, the motorized gantry stage, and the second motorized gantry stage can all be disposed within a G-arm fluoroscopic system.

In accordance with aspects of the present invention, the non-parallax panoramic image is stitched together based on identifying correlations between adjacent images established from mechanical position of the radiation detector attached to the motorized gantry stage. The non-parallax panoramic image can be stitched together based on identifying overlapping fields of view. The stitching can be performed by the processing and display device in real time. A viewpoint of the non-parallax panoramic image can be provided by a fixed focal spot provided by the dynamic collimator.

In accordance with aspects of the present invention, the processing and display device performs the stitching by applying a weighting profile. The weighting profile can be one of triangular and Gaussian.

In accordance with example embodiments of the present invention, method for utilization of a fluoroscopic imaging system is provided. The method includes activating a fluoroscopic imaging device. The fluoroscopic imaging device includes a radiation source, a dynamic collimator coupled to the radiation source that focuses electromagnetic radiation provided by the radiation source at a radiation detector, and a radiation detector coupled to a motorized gantry stage and disposed to read electromagnetic radiation output by the radiation source, wherein the radiation detector changes position based on a position of the motorized gantry stage. The fluoroscopic imaging device also includes a processing and display device configured to receive raw image data, the raw image data comprising a plurality of images captured at the position of the motorized gantry stage relative a subject patient located between the radiation source and the radiation detector. The method also includes receiving, by the processing and display device, raw image data comprising a plurality of images captured at various positions of the motorized gantry stage and radiation detector and transforming the raw image data, by the processing and display device, into displayable images of the subject patient. The method further includes stitching, by the processing and display device, together the displayable images, based on the position of the motorized gantry stage, into a non-parallax panoramic image and displaying, by the processing and display device, the non-parallax panoramic image in real time.

In accordance with aspects of the present invention, the system further includes performing a fluoroscopic procedure relying on the non-parallax panoramic image. The fluoroscopic imaging device reduces a dosage applied to the subject patient.

### BRIEF DESCRIPTION OF THE FIGURES

These and other characteristics of the present invention will be more fully understood by reference to the following detailed description in conjunction with the attached drawings, in which:

FIG. 1A is an illustration depicting the main components of a conventional G-arm medical imaging system;

FIG. 1B is an example illustration of a conventional imaging system;

FIG. 2 is a diagrammatic illustration of a panoramic imaging system, in accordance with embodiments of the present invention;

FIG. 3A is a diagrammatic illustration of the operation of an imaging system to produce a non-parallax panoramic image from a plurality of individual overlapping images, in accordance with aspects of the present invention;

FIG. 3B is a diagrammatic illustration of the operation of an imaging system to produce a non-parallax panoramic image from a plurality of individual overlapping images, in accordance with aspects of the present invention;

FIG. 4 is a flowchart depicting an example operation of the imaging system, in accordance with aspects of the present invention; and

FIG. 5 is a diagrammatic illustration of a high level architecture for implementing processes in accordance with aspects of the present invention.

### DETAILED DESCRIPTION

An illustrative embodiment of the present invention relates to a method and system for combining individual overlapping medical images into a single undistorted panoramic image in real-time. The present invention utilizes a combination of a dynamic collimator attached to a radiation source and a radiation detector attached to a motorized gantry stage to create the panoramic image from a collection of individual images. In particular, the present invention utilizes the dynamic collimator to direct radiation produced by the radiation source toward a moving radiation detector (e.g., in motion via the motorized gantry stage). Based on the known position of the radiation detector when limited field of view images are captured, the present invention identifies overlapping fields of view between a plurality of images, such that the overlaps can be used in a digital stitching process to create a digital panoramic image. Specifically, the present invention utilizes a mechanical positioning methodology in which the motion of motorized radiation detector is used to determine the image translation between the individual images and stitches the images together to form the panoramic image based on the image translation.

The combination of elements utilized in the present invention provides an optimized stitching implementation that is fast enough for real-time stitching and displaying of a digital panoramic image of a patient while the image receptor is moving along the patient. Additionally, the present invention produces robust and accurate panoramic images with quality and spatial resolution that is comparable to that of the individual images, without the utilization of down-sampling and masking. The present invention, however can utilize down-sampling and masking to further optimize and increase the speed of the stitching process, if desired. The combination of benefits and functionality provided by the present invention make the invention ideal for use in real-time during a fluoroscopic procedure. The real-time panoramic images provided by the present invention improve the effectiveness, reliability, and accuracy of the user performing the fluoroscopic procedure. Moreover, the radiation steered by the dynamic collimation reduces dosage and x-ray scattering inside patient body during the procedure.

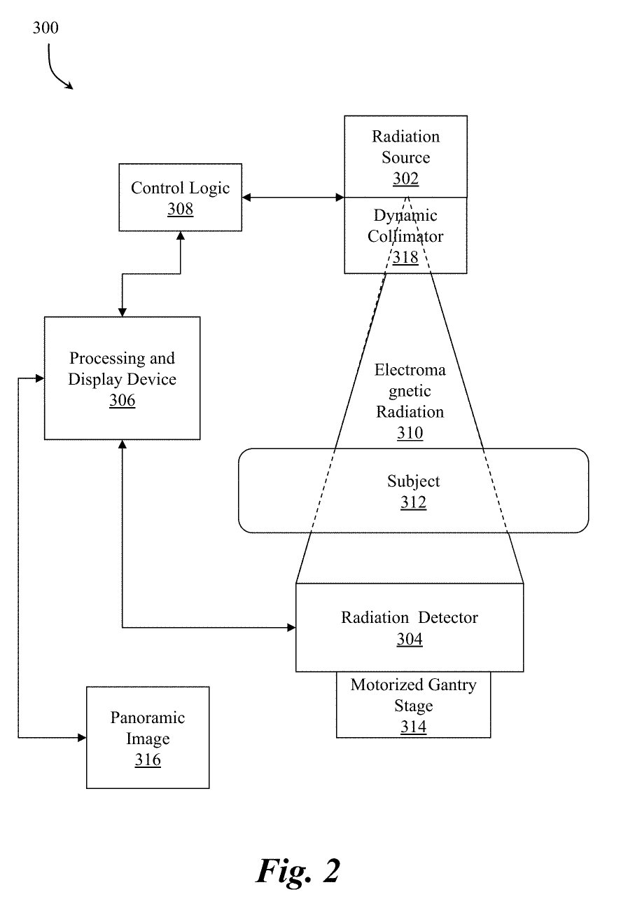

FIGS. 2 through 5, wherein like parts are designated by like reference numerals throughout, illustrate an example embodiment or embodiments of an improved system for creating real-time panoramic images during a fluoroscopic procedure, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

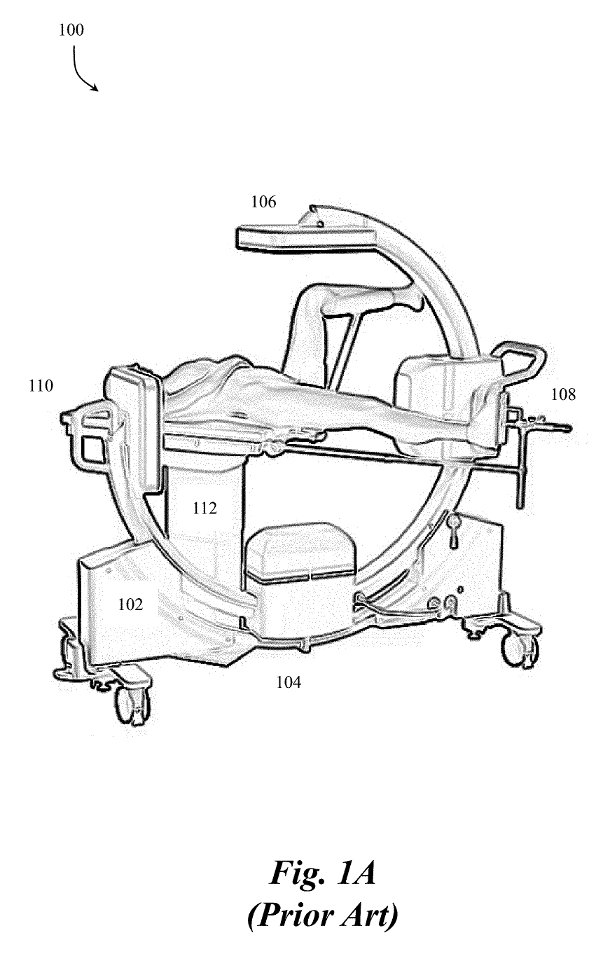

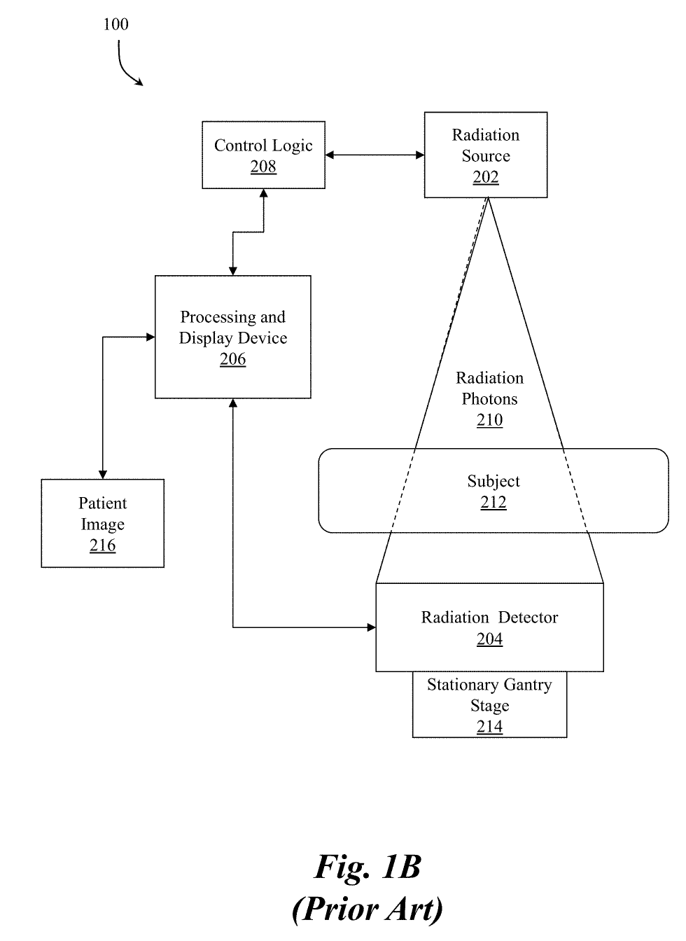

Traditionally, fluoroscopic imaging procedures can be implemented utilizing a collection of different imaging systems (e.g., C-arm, G-arm bi-plane fluoroscopic imager, etc.). An example of an imaging system is depicted in FIG. 1A. In particular, FIG. 1A depicts the main components of a G-arm medical imaging system 100 which can be utilized during a fluoroscopic procedure. The main components of the G-arm system include a movable stand 102, a radiation source 104 and radiation detector 106 configured for a frontal view (or anteroposterior view), a radiation source 108 and radiation detector 110 configured for a lateral view, and a patient table 112 configured to hold a patient between the radiation sources 104, 108 and the radiation detectors 106, 110. As would be appreciated by one skilled in the art, the radiation sources 104, 108 can include any kind of radiation sources utilized for imaging a patient. For example, the radiation sources 104, 108 can be electromagnetic radiation or x-radiation sources configured to produce X-rays.

FIG. 1B depicts a diagrammatic illustration of a conventional imaging system 200 that can be utilized during a fluoroscopic procedure. For example the system 200 could be utilized in the configuration provided in FIG. 1A (e.g., a G-arm configuration) or in alternative configurations (e.g., a C-arm configuration). In particular, FIG. 1B depicts the traditional radiation detection systems 200 (e.g., X-ray photon detection system) configured for a single plane imaging applications or one plane of bi-plane imaging applications. An example of a system that would be configured for a single plane imaging application is a C-arm device. The radiation detection system 200, as depicted in FIG. 1B, includes a radiation source device 202 (e.g., X-ray source), radiation detector 204 (or fluoroscopic imager or X-ray photon detector), a processing and display device 206, and a control logic device 208. As depicted in FIG. 1B, the combination of elements 202, 204, 206, 208 are configured to be attached to a gantry of a C-arm device. Additionally, typically, the flat panel radiation detector 204 is attached to the gantry of a C-arm device via a stationary gantry stage 214. As would be appreciated by one skilled in the art, the radiation source 202 is a device configured to produce radiation 210 (e.g., X-ray photons) for projection through a subject patient 212 (e.g., a patient) positioned on a patient table.

Similarly, as would be appreciated by one skilled in the art, the conventional radiation detection system 200 provided in FIG. 1B can be configured for use with a G-arm device. In particular, the G-arm device would utilize two radiation sources 202 and two radiation detectors 204 attached to a stationary gantry stage(s) 214 in a configuration to simultaneously the capture a posterior image of a patient and a lateral position of the patient (e.g., perpendicular sources and detectors as shown in FIG. 1A). For example, the radiation detection system 200 discussed with respect to FIG. 1B could be implemented in the configuration shown on the G-arm device system 100 discussed with respect to FIG. 1A to capture bi-plane images of a patient 214.

Continuing with FIG. 1B, the control logic 208 is configured to receive input from the processing and display device 206 (e.g., via an input from a user) and transmit signals to control the radiation source 202. In particular, the control logic 208 provides signals for operating the radiation source 202 and when to produce radiation 210. The radiation detector 204 is configured to electrically transform received radiation 210, produced by the radiation source 202, into detectable signals. An example of a traditional radiation detector 106 is a flat panel detector, which is a thin film transistor (TFT) panel with a scintillation material layer configured to receive energy from visible photons to charge capacitors of pixel cells within the TFT panel. The charges for each of the pixel cells are readout as a voltage data value to the processing and display device 206 as an image 216 of the patient (e.g., an X-ray image). As would be appreciated by one skilled in the art, each of the components within the conventional radiation detection system 200 can include a combination of devices known in the art configured to perform the imaging tasks discussed herein. For example, an image intensifier is an alternative radiation detector that can be utilized in place of the radiation detector 204 system.

FIG. 2 depicts a diagrammatic illustration of an imaging system for use in accordance with the present invention. In particular, FIG. 2 depicts a panoramic fluoroscopic imaging system 300 configured to capture panoramic images of a patient in real-time during a fluoroscopic procedure. The fluoroscopic imaging system 300 can be implemented using a combination of imaging devices including, but not limited to, a C-arm or G-arm device. The fluoroscopic imaging system 300, as depicted in FIG. 2, includes a radiation source 302 (e.g., X-ray source) configured to output electromagnetic radiation 310 (e.g., X-ray photons), a radiation detector 304 (or fluoroscopic imager or X-ray photon detector) coupled to a motorized gantry stage 314 and disposed to read electromagnetic radiation 310 output by the radiation source 302, a dynamic collimator 318 coupled to the radiation source 302 that focuses the electromagnetic radiation 310 output by the radiation source 302 and directs the focused electromagnetic radiation 310 at the radiation detector 304, a control logic device 308, and a motorized gantry stage 314.

The radiation source 302 is a device configured to produce radiation 310 (e.g., X-ray photons) for projection through a subject patient 312 (e.g., a patient) positioned on a patient table to the radiation detector 304. In accordance with an example embodiment of the present invention, the radiation detector 304 is a thin film transistor (TFT) flat-panel detector with a scintillation material layer. When the TFT receives energy from visible photons that charge capacitors of pixel cells within the TFT panel, charges from each of the pixel cells are readout as a voltage data value to a processing and display device 306. As would be appreciated by one skilled in the art, the radiation detector can also be an image intensifier configured to readout a voltage data value to a processing and display device 306.

Continuing with FIG. 2, the combination of elements 302, 304, 306, 308 are configured to be attached to a gantry of a C-arm or G-arm device. In accordance with an example embodiment of the present invention, the radiation detector 304 is attached to the gantry of a C-arm or G-arm device via the motorized gantry stage 314. As would be appreciated by one skilled in the art, the fluoroscopic imaging system 300 implemented on a G-arm would include a second radiation source 302, a second radiation detector 304 coupled to a second motorized gantry stage 314, and a second dynamic collimator 318. Therefore the G-arm would include the radiation source 302, the second radiation source 302, the first radiation detector 304, the second radiation detector 304, the first dynamic collimator 318, the second dynamic collimator 318, the first motorized gantry stage 314, and the second motorized gantry stage 314 all disposed within a G-arm fluoroscopic gantry.

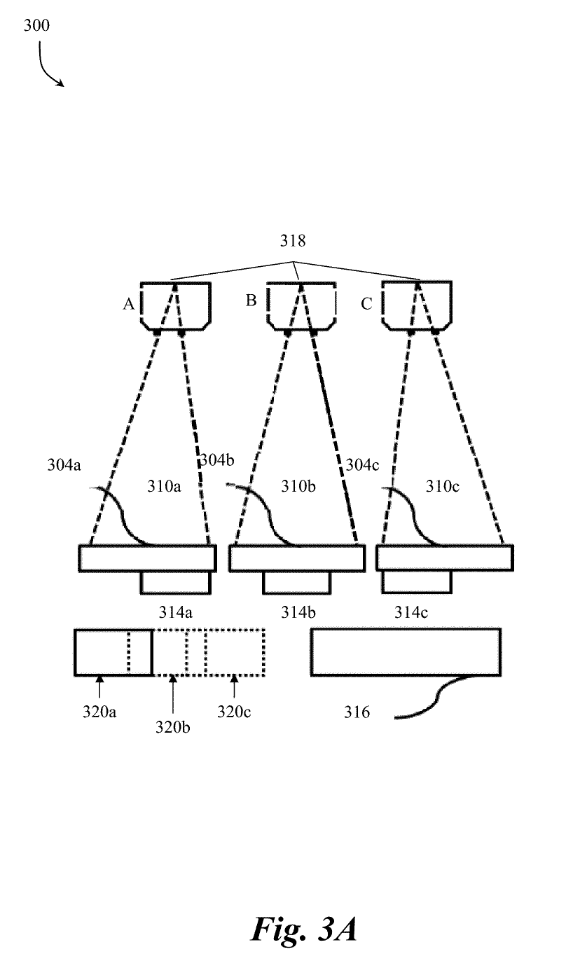

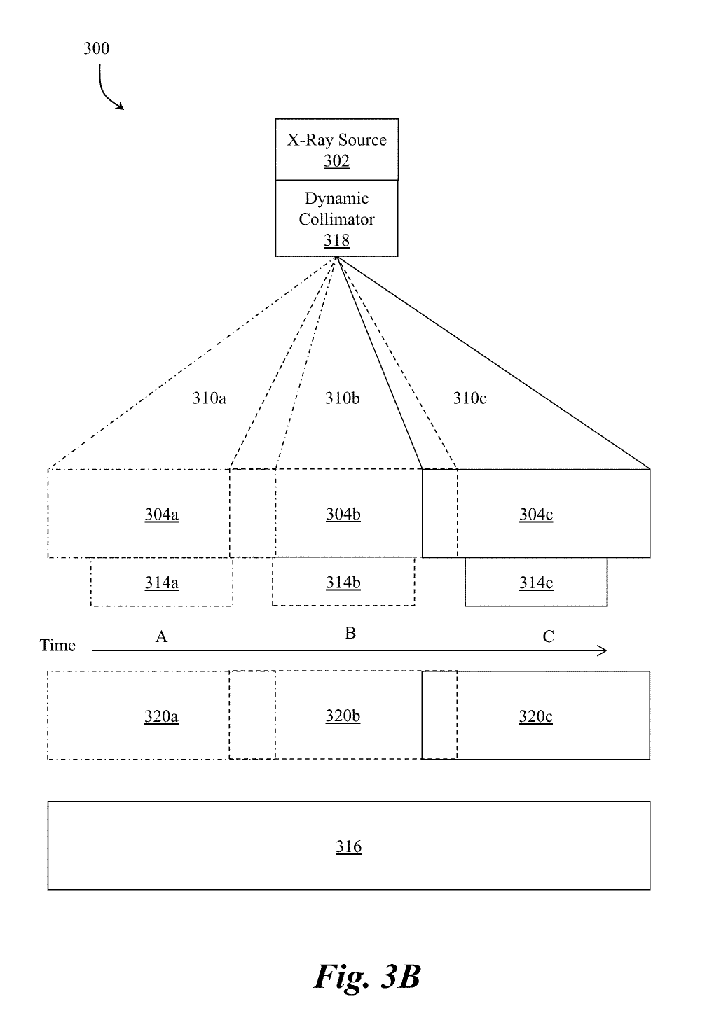

Additionally, the fluoroscopic imaging system 300 includes or is otherwise in communication with the processing and display device 306. In accordance with an example embodiment of the present invention, the processing and display device 306 is configured to receive raw image data from the radiation detector 304, the raw image data including a plurality of limited field of view images 320a, 320b, 320c captured at various locations on a subject patient 312 located between the radiation source 302 and the radiation detector 304. In particular, the processing and display device 306 receives the plurality of images 320a, 320b, 320c captured by the radiation detector 304 resulting from the radiation detector 304 being transported to different locations via the motorized gantry stage 314. The processing and display device 306 transforms the raw image data for each of the plurality of images 320a, 320b, 320c into displayable images, stitches together the displayable images into a non-parallax panoramic image 316, and displays the non-parallax panoramic image 316 on the display device in real time. In accordance with an example embodiment of the present invention, the plurality of images 320a, 320b, 320c are stitched together based on the positions of the radiation detector 304 (as transported by the motorized gantry stage 314) when the images 320a, 320b, 320c were captured, as discussed in greater detail with respect to FIGS. 3A and 3B.

In operation, the fluoroscopic imaging system 300 is configured to capture a plurality of independent limited field of view and overlapping images 320a, 320b, 320c and transform the overlapping images 320a, 320b, 320c into a single undistorted non-parallax panoramic image 316 that is the equivalent of a single image. Although the operation of the present invention is discussed with respect to a single radiation source 302 and radiation detector 304 to produce a single plane image (e.g., a C-arm implementation), as would be appreciated by one skilled in the art, the fluoroscopic imaging system 300 can also utilize multiple radiation sources 302 and radiation detectors to produce bi-plane images (e.g., a G-arm implementation) without departing from the scope of the present invention. The fluoroscopic imaging system 300 begins the creation of the panoramic image 316 by initiating the radiation source 302 to generate radiation 310 through a patient 312 to be received by a radiation detector 304. During the generation of radiation 310 by the radiation source 302, the dynamic collimator 318 focuses and directs the radiation 310 at a specified location. In particular, the dynamic collimator 318 directs the radiation 310 toward a location of the radiation detector 304.

In accordance with an example embodiment of the present invention, during generation of the radiation 310, the motorized gantry stage 314 (and the radiation detector 304 attached thereto) traverses in two directions along a fixed track situated on a path parallel to the radiation source 302 and dynamic collimator 318. As the motorized gantry stage 314 traverses, with the radiation detector 304 attached thereto, the dynamic collimator 318 will redirect the radiation 310 such that the radiation is continuously focused and directed to the location of the radiation detector 304. While the radiation detector 304 traverses via the motorized gantry stage 314 and the dynamic collimator 318 directs the radiation 310, the processing and display device 306 receives raw image data from the radiation detector 304. As would be appreciated by one skilled in the art, the raw data can be periodically sampled to create data for the plurality of independent images 320a, 320b, 320c. In accordance with an example embodiment of the present invention, each transmission of each independent collection of raw data (e.g., for each individual image) includes a respective location of the radiation detector 304 (e.g., according to a mechanical positioning of the motorized gantry stage) at the time that the raw data was captured.

Thereafter, the processing and display device 306 transforms each independent collection of raw data into a digital image to create a plurality of limited field of view images 320a, 320b, 320c. In accordance with an example embodiment of the present invention, the image data is sampled such that the captured plurality of images 320a, 320b, 320c are overlapping images. Utilizing the received mechanical position of the radiation detector 304 and/or the motorized gantry stage 314, the processing and display device 306 creates a single non-parallax wide-view panoramic image 316 by stitching together the overlapping plurality of images 320a, 320b, 320c. In accordance with an example embodiment of the present invention, the non-parallax panoramic image 316 is stitched together based on identifying correlations between adjacent images 320a, 320b, 320c established from mechanical position of the radiation detector 304 attached to the motorized gantry stage 314. In particular, the panoramic image 316 is created by identifying the overlapping regions of the plurality of images 320a, 320b, 320c from the mechanical movement/positioning and interpolating the images from an adjacent view utilizing a weighting profile. For example, the processing and display device 306 can utilize a Gaussian or triangular weighting profile to create the panoramic image 316. Additionally, because the radiation 310 is focused and directed from a single point (e.g., the dynamic collimator 318), the non-parallax panoramic image 316 is created with a fixed focal point of the dynamic collimator 318. Once the panoramic image 316 is created, the processing and display device 306 can display the image to a user in real-time (e.g., for use during a fluoroscopic procedure).

In accordance with an example embodiment of the present invention, the stitching method to produce the panoramic image 316 is fully automated without any user input required. As would be appreciated by one skilled in the art, the stitching can be performed utilizing any stitching methods and systems known in the art to combine a plurality of images into a single image (e.g., through interpolating, blending, etc.). The stitching image frames together and displaying the stitched panoramic image 316 in real-time while the radiation detector 304 is moving along the patient, however, may require a user to control the radiation detector 304 moving, acquiring, and stopping (e.g., via the data processing and display device 306).

FIGS. 3A and 3B depict example implementations of the fluoroscopic imaging system 300 for use in accordance with the present invention. In particular, FIGS. 3A and 3B depict exemplary representations of how each of the components in the fluoroscopic imaging system 300 operates to create non-parallax panoramic images 316. FIG. 3A depicts an example representation of the operation of the fluoroscopic imaging system 300 to produce a non-parallax panoramic image 316 from a plurality of individual overlapping images 320a, 320b, 320c captured at different mechanical locations via the motorized gantry stage 314. In particular, FIG. 3A depicts the fluoroscopic imaging system 300 at three different points in time during operation (e.g., during a fluoroscopic procedure) to capture a plurality of images 320a, 320b, 320c to be transformed into a panoramic image 316. At a first point in time A, the motorized gantry stage 314a, with the radiation detector 304a attached thereto, is located at a first position and the dynamic collimator 318 focuses and directs the radiation 310a toward the first location of the radiation detector 304a. When the motorized gantry stage 314a is located at the first location, the radiation detector 304a captures and transmits the raw image data (resulting from exposure to the radiation 310a) to the processing and display device 306 for transformation into a displayable image 320a. Simultaneous with the radiation detector 304a transmitting the raw image data, the position of motorized gantry stage 314a is captured and transmitted to the processing and display device 306.

At a second point in time B, the motorized gantry stage 314b, with the radiation detector 304b attached thereto, traverses to a second location and the dynamic collimator 318 focuses and directs the radiation 310b toward the second location of the radiation detector 304b. When the motorized gantry stage 314b is located at the first location, the radiation detector 304b captures and transmits the raw image data (resulting from exposure to the radiation 310b) to the processing and display device 306 for transformation into a displayable image 320b. Simultaneous with the radiation detector 304b transmitting the raw image data, the position of motorized gantry stage 314b is captured and transmitted to the processing and display device 306.

At a third point in time C, the motorized gantry stage 314c, with the radiation detector 304c attached thereto, traverses to a third location and the dynamic collimator 318 focuses and directs the radiation 310c toward the second location of the radiation detector 304c. When the motorized gantry stage 314c is located at the first location, the radiation detector 304c captures and transmits the raw image data (resulting from exposure to the radiation 310c) to the processing and display device 306 for transformation into a displayable image 320c. Simultaneous with the radiation detector 304c transmitting the raw image data, the position of motorized gantry stage 314c is captured and transmitted to the processing and display device 306.

Continuing with FIG. 3A, once each of the displayable images 320a, 320b, 320c is transformed by the processing and display device 306, the non-parallax panoramic image 316 is created. In particular, the processing and display device 306 utilizes the respective captured positions of the motorized gantry stage 314a, 314b, 314 to identify the overlapping fields of view of the images 320a, 320b, 320c. For example, the processing and display device 306 has the dimensions of each image 320a, 320b, 320b, and where each image was located during the image capture, and thus, the processing and display device 306 can resolve what positions of those images are overlapping one another (e.g., calculating linear translation distances). Thereafter, the processing and display device 306 creates the non-parallax panoramic image 316 by stitching together the images 320a, 320b, 320b based on the identified overlapping. Additionally, the processing and display device 306 performs the stitching by applying a weighting blending profile (e.g., triangular or Gaussian weighting). The weighted blending is the contribution factor of a pixel in a sub-image to panoramic/stitching image. Utilizing the above-noted methodology and system, the fluoroscopic imaging system 300 is able to produce the single non-parallax panoramic image 316 provides data for use during a fluoroscopic procedure. As would be appreciated by one skilled in the art, although the plurality of images 320a, 320b, 320c are referred to in the example implementations as three images, any number of images could be utilized without departing from the scope of the present invention. The utilization of the three images 320a, 320b, 320c is for explanation purposes only and not intended to limit the present invention to the utilization of three images as depicted in FIGS. 3A and 3B.

FIG. 3B depicts an example of the operation of the fluoroscopic imaging system 300 to produce a non-parallax panoramic image 316 from a plurality of individual overlapping images 320a, 320b, 320c captured at different mechanical locations via the motorized gantry stage 314. In particular, FIG. 3B depicts another representation of the fluoroscopic imaging system 300 performing the same operation discussed with respect to FIG. 3A. More specifically, FIG. 3B depicts a plurality of images 320a, 320b, 320c and the respective positions of the motorized gantry stage 314a, 314b, 314c and radiation detector 304a, 304b, 304c at different points in time A, B, C for capturing those images 320a, 320b, 320c. Additionally, FIG. 3B depicts how the x-ray source 302, the dynamic collimator 318, the motorized gantry stage 314, and the radiation detector 304 are utilized to capture the plurality of images 320a, 320b, 320c which are utilized to create the single non-parallax panoramic image 316.

At a first point in time A, the motorized gantry stage 314 is located at a first location 314a (with the radiation detector 304 attached thereto) and the dynamic collimator 318 focuses and directs the electromagnetic radiation 310, produced by the x-ray source 302, at the first location 304a of the radiation detector 304 on the motorized gantry stage 314 (at the first location 314a) to create an electromagnetic radiation beam 310a. When the motorized gantry stage 314 is located at the first location 314a and the electromagnetic radiation beam 310a is created at the location 304a of the radiation detector 304, the radiation detector 304 captures the first image 320a.

Thereafter, the motorized gantry stage 314 traverses to a second location 314b at point in time B and the dynamic collimator 318 simultaneously re-focuses and re-directs the electromagnetic radiation 310 to the location 304b of the radiation detector 304 to create an electromagnetic radiation beam 310b. When the motorized gantry stage 314 is located at the second location 314b and the electromagnetic radiation beam 310b is created at the ocation 304b of the radiation detector 304, the radiation detector 304 captures the second image 320b. This process repeats for point in time C, in which the motorized gantry stage 314 traverses to a third location 314c and the dynamic collimator 318 focuses and directs an electromagnetic radiation beam 310c at a location 304c of the radiation detector 304 that captures a third image 320c. As would be appreciated by one skilled in the art, the process discussed with respect to FIG. 3B can be repeated for any N number of images at N number of locations, and the present invention is not intended to be limited to capturing three images at three locations over three points in time.

Continuing with FIG. 3B, once each of the captured images 320a, 320b, 320c is received by the processing and display device 306, the images 320a, 320b, 320c are transformed into the non-parallax panoramic image 316 (e.g., by stitching together the images 320a, 320b, 320c), as discussed herein with respect to FIGS. 2 and 3A. Utilizing the above-noted methodology and system, the fluoroscopic imaging system 300 is able to produce the single non-parallax panoramic image 316 from the plurality of images 320a, 320b, 320c sharing a single focal point (e.g., the dynamic collimator 318).

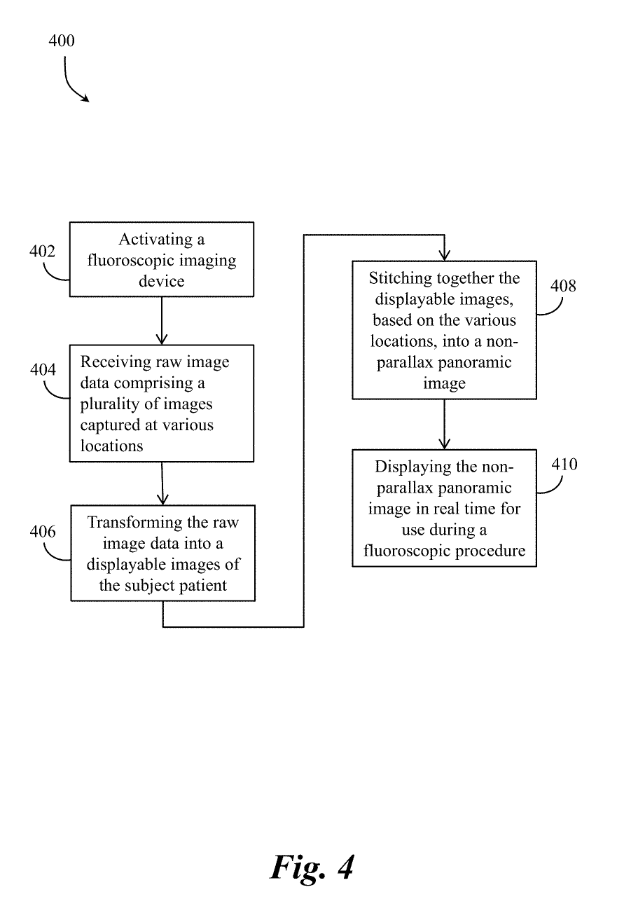

FIG. 4 depicts an example operation of the fluoroscopic imaging system 300 in accordance with the present invention. In particular, FIG. 4 depicts a process 400 operation for utilization of a fluoroscopic imaging system. At step 402 a fluoroscopic imaging system (e.g., a fluoroscopic imaging system 300 as discussed with respect to FIGS. 2, 3A, and 3B) is activated. At step 404 a processing and display device (e.g., display device 306) receives raw image data including a plurality of limited field of view images (e.g., images 320a, 320b, 320c), each captured at the position of the motorized gantry stage 314 relative a subject patient located between the radiation source and the radiation detector. Additionally, the position of the motorized gantry stage during the image capture is obtained by the processing and display device. At step 406 the processing and display device transforms the raw image data into displayable images of the subject patient. At step 408 the processing and display device stitches together the displayable images, based on the position of the motorized gantry stage, into a non-parallax panoramic image 316. At step 410 the processing and display device displays the non-parallax panoramic image (e.g., panoramic image 316) to a user in real time (e.g., for use during a fluoroscopic procedure). Relying on the real-time panoramic image, the user can perform a fluoroscopic procedure, which reduces a radiation dosage applied to the patient.

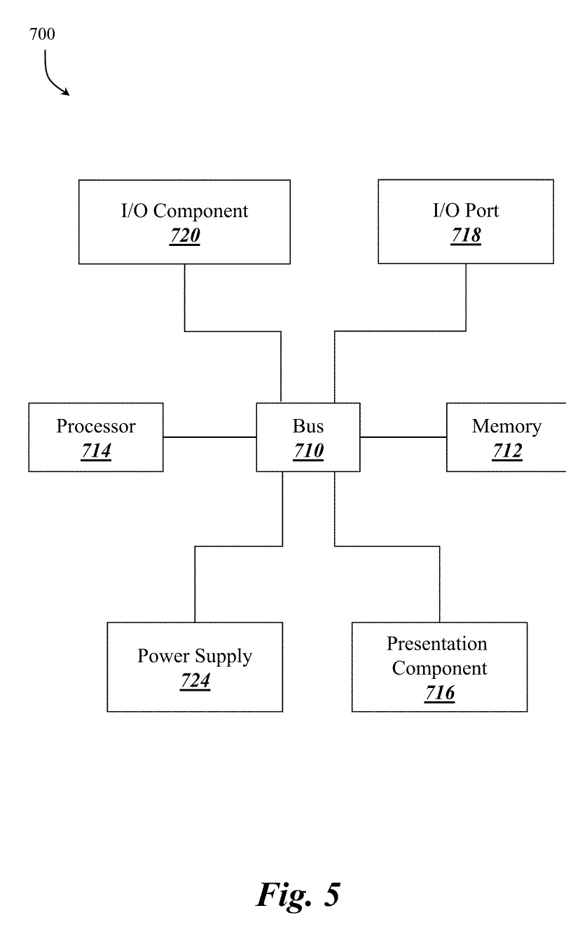

Any suitable computing device can be used to implement the computing devices (e.g., processing and display device 306) and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 700 is depicted in FIG. 5. The computing device 700 is merely an illustrative example of a suitable computing environment and in no way limits the scope of the present invention. A “computing device,” as represented by FIG. 5, can include a “workstation,” a “server,” a “laptop,” a “desktop,” a “hand-held device,” a “mobile device,” a “tablet computer,” or other computing devices, as would be understood by those of skill in the art. Given that the computing device 700 is depicted for illustrative purposes, embodiments of the present invention may utilize any number of computing devices 700 in any number of different ways to implement a single embodiment of the present invention. Accordingly, embodiments of the present invention are not limited to a single computing device 700, as would be appreciated by one with skill in the art, nor are they limited to a single type of implementation or configuration of the example computing device 700.

The computing device 700 can include a bus 710 that can be coupled to one or more of the following illustrative components, directly or indirectly: a memory 712, one or more processors 714, one or more presentation components 716, input/output ports 718, input/output components 720, and a power supply 724. One of skill in the art will appreciate that the bus 710 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 5 is merely illustrative of an exemplary computing device that can be used to implement one or more embodiments of the present invention, and in no way limits the invention.

The computing device 700 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 700.

The memory 712 can include computer-storage media in the form of volatile and/or nonvolatile memory. The memory 712 may be removable, non-removable, or any combination thereof. Exemplary hardware devices are devices such as hard drives, solid-state memory, optical-disc drives, and the like. The computing device 700 can include one or more processors that read data from components such as the memory 712, the various I/O components 716, etc. Presentation component(s) 716 present data indications to a user or other device. Exemplary presentation components include a display device, speaker, printing component, vibrating component, etc.

The I/O ports 718 can enable the computing device 700 to be logically coupled to other devices, such as I/O components 720. Some of the I/O components 720 can be built into the computing device 700. Examples of such I/O components 720 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A panoramic fluoroscopic imaging system, comprising:
a radiation source configured to output electromagnetic radiation;
a radiation detector coupled to a motorized gantry stage and disposed to read electromagnetic radiation output by the radiation source;
a dynamic collimator coupled to the radiation source that focuses the electromagnetic radiation output by the radiation source and directs the focused electromagnetic radiation at the radiation detector, wherein the radiation detector changes position based on a position of the motorized gantry stage;
a processing and display device in communication with the fluoroscopic imaging device, the processing and display device configured to:
receive raw image data from the radiation detector, the raw image data comprising a plurality of images captured at the position of the motorized gantry stage relative to a subject patient located between the radiation source and the radiation detector;
transform the raw image data for each of the plurality of images into displayable images;
stitch together the displayable images, based on the position of the motorized gantry stage, into a non-parallax panoramic image; and
display the non-parallax panoramic image on the display device in real time.

2. The system of claim 1, wherein the radiation detector comprises a thin film transistor (TFT) flat-panel detector with a scintillation material layer.

3. The system of claim 2, wherein when the TFT receives energy from visible photons that charge capacitors of pixel cells within the TFT panel, charges from each of the pixel cells are readout as a voltage data value to the processing and display device.

4. The system of claim 1, wherein the radiation detector comprises an image intensifier configured to readout a voltage data value to the processing and display device.

5. The system of claim 1, wherein the single non-parallax panoramic image provides data for use during a fluoroscopic procedure.

6. The system of claim 1, wherein the radiation source, the radiation detector, the dynamic collimator, and the motorized gantry stage are all disposed within a C-arm fluoroscopic system.

7. The system of claim 1, further comprising:
a second radiation source;
a second radiation detector coupled to a second motorized gantry stage; and
a second dynamic collimator.

8. The system of claim 7, wherein the radiation source, the second radiation source, the radiation detector, the second radiation detector, the dynamic collimator, the second dynamic collimator, the motorized gantry stage, and the second motorized gantry stage are all disposed within a G-arm fluoroscopic system.

9. The system of claim 1, wherein the non-parallax panoramic image is stitched together based on identifying correlations between adjacent images established from mechanical position of the radiation detector attached to the motorized gantry stage.

10. The system of claim 1, wherein the non-parallax panoramic image is stitched together based on identifying overlapping fields of view.

11. The system of claim 1, wherein the stitching is performed by the processing and display device in real time.

12. The system of claim 1, wherein a viewpoint of the non-parallax panoramic image is provided by a fixed focal spot provided by the dynamic collimator.

13. The system of claim 1, wherein the processing and display device performs the stitching by applying a weighting profile.

14. The system of claim 13, wherein the weighting profile is one of triangular and Gaussian.

15. A method for utilization of a fluoroscopic imaging system, the method comprising:
activating a fluoroscopic imaging device comprising:
a radiation source;
a dynamic collimator coupled to the radiation source that focuses electromagnetic radiation provided by the radiation source at a radiation detector;
a radiation detector coupled to a motorized gantry stage and disposed to read electromagnetic radiation output by the radiation source, wherein the radiation detector changes position based on a position of the motorized gantry stage; and
a processing and display device configured to receive raw image data, the raw image data comprising a plurality of images captured at the position of the motorized gantry stage relative a subject patient located between the radiation source and the radiation detector;
receiving, by the processing and display device, raw image data comprising a plurality of images captured at various positions of the motorized gantry stage and radiation detector;
transforming the raw image data, by the processing and display device, into displayable images of the subject patient;
stitching, by the processing and display device, together the displayable images, based on the position of the motorized gantry stage, into a non-parallax panoramic image; and
displaying, by the processing and display device, the non-parallax panoramic image in real time.

16. The method of claim 15, further comprising performing a fluoroscopic procedure relying on the non-parallax panoramic image.

17. The method of claim 15, wherein the fluoroscopic imaging device reduces a dosage applied to the subject patient.
